(19) United States
(12) Patent Application Publication
(10) Pub. No.: US 2006/0189909 A1
(43) Pub. Date: Aug. 24, 2006

(54) Load relieving wound dressing

(76) Inventors: Timothy B. Hurley, Boalsburg, PA (US); Jan S. Ulbrecht, Boalsburg, PA (US)

Correspondence Address: JEANNE E. LONGMUIR, 2836 CORYDON ROAD, CLEVELAND HEIGHTS, OH, 44118, US

(21) Appl. No.: 11/065,469

(22) Filed: Feb. 24, 2005

(51) Int. Cl. A61F 15/00; A61F 13/00

(52) U.S. Cl. 602/41; 602/42

(57) ABSTRACT

An improved load relieving dressing for transferring pressure away from a wound on a foot of a patient. The dressing having a wound opening formed through the dressing for surrounding a wound site. The dressing having an external shape which substantially conforms to the shape of the pressure distribution on the foot during load bearing. A first layer of the dressing interfacing with and cushioning a foot during load bearing. The first layer is of a material sufficient to limit pressure increases at the wound opening and within said dressing adjacent the wound opening. An off-loading layer is engaged with said first layer, and is of a material sufficiently thick and firm to reduce pressure at the wound site.

### TECHNICAL FIELD

The present application is directed to an improved wound dressing, and more specifically to a multi-layered wound dressing for maximizing the pressure or load transferred from a wound site during load bearing.

### BACKGROUND

Mechanical off-loading is an essential factor in the healing of foot plantar surface ulcers. The medical condition of peripheral neuropathy in diabetic patients leads to the loss of sensation such that skin injury and complete breakdown, resulting in ulcers, can develop with no or minimal pain. These wounds tend not to heal because of ongoing mechanical trauma not felt at all by the patient as painful. Such wounds can only be healed by treatment which includes protecting them from mechanical trauma. Methods for healing plantar ulcers include providing a total contact cast for the foot, which provides substantial mechanical protection. This method is not ideal for application in the health care practice settings of primary care practitioners and endocrinologists, where such wounds are normally treated, because it requires skilled and specialized care in application, along with frequent follow up, and is time consuming to apply. Also, patients perceive the cast to be an inconvenience at the early stages of such a wound, which is often perceived by them as not a serious matter. An alternative healing method is to ask the patient to follow a non-weight bearing regimen through the use of a wheelchair, crutches, or a walker. Such complete mechanical protection is possible only with full patient compliance. This alternative rarely proves to be effective in healing wounds within a reasonable time period, because compliance is extremely poor. A still further alternative to unloading pressure from the wound is discussed in U.S. Pat. Nos. 6,610,897 and 6,720,470. While such wound healing systems provide an improvement over conventional cast and non-weight bearing methods, they are often difficult for medical personnel to properly form, fit and apply to a patient.

A new alternative has been developed which further reduces plantar foot pressure at a wound site, is convenient for application by medical personnel, and which is accepted by the patient.

### BRIEF SUMMARY OF THE INVENTION

An improved load-relieving dressing (“LRD”) and improved foot bed have been developed and have been shown to increase mechanical off-loading performance by reducing foot plantar surface pressure at the wound site during load bearing. The dressing and foot bed combination of the present application are simple for health care practitioners to apply, easy for patients to use and create a more favorable environment for wound healing.

Improved Load Relieving Dressing

Improved features of the load-relieving dressing include dressing geometry, incorporation of new off-loading materials, multiple material layers, dressing thickness and use of the dressing within an improved foot bed or other foot gear which do not require the provider to make custom modifications.

During development of the present LRD it was learned that dressing geometry, or the shape of the dressing, plays an important role in providing a contact area for the remainder of the patient's foot surface. The LRD of the present application is shaped to correspond approximately and substantially to the typical load bearing regions of the foot surface, for example in the forefoot. Concentration of pressure in these regions is a contributing risk factor for the development of foot ulcers in patients diagnosed with diabetic neuropathy. By tailoring the geometry of the LRD to match the foot region of interest, the present invention effectively maximizes the contact area available to transfer load away from the wound site and thereby reduce plantar pressure.

Different geometries of dressings may be provided for different areas or regions of the foot. Preferably dressings are provided for the metatarsal head region, the heel region and the hallux region, since these are the typical load bearing regions of the foot plantar surface and, subsequently, are at the greatest risk for development of foot ulcers. A wound opening, or off-loading aperture(s), may be provided in each dressing. The location and diameter of the wound opening(s) may be varied based upon the presentation of the wound or wound sites. The LRDs for each of the regions cover essentially all of the regional weight-bearing anatomy of the foot. For example, the LRD for the metatarsal head region is positioned under the forefoot and covers essentially all of the load bearing portion of the forefoot, except the wound site, to maximize distribution of forefoot plantar pressure.

Specifically, the geometry of the metatarsal head region has a configuration which is designed to engage, or substantially engage, the pressure distribution on the foot during load bearing. In the design of the present application, the load relieving dressing for the metatarsal head region may be used on either the left forefoot or the right forefoot, since rotation of the dressing enables alignment of the dressing with the pressure distribution surface of either forefoot region. The heel and hallux region LRDs may also be interchanged between the right foot and left foot. In the metatarsal head region, the LRD functions much like an insole and enables the patient to walk more normally when worn alone.

In order to minimize plantar pressure increases in areas adjacent to or surrounding the wound during load bearing, the LRD of the present application includes multiple material layers. Each of the layers is of a material for accomplishing a specific purpose. Additionally, the thickness of each of the layers may be varied. A cushioning layer is provided which interfaces with the plantar surface of the foot. The cushioning layer serves to reduce and/or limit pressure increases at the periphery of the wound opening or off-loading aperture of the LRD and in the immediate area of the LRD adjacent to the wound site. In the non-load bearing condition, the cushioning layer at the periphery of the wound opening may have a substantially square edge portion, which eases under pressure.

An off-loading layer is provided which is secured to the cushioning layer on a surface away from the foot. The off-loading layer is of a thickness and firmness sufficient to maximize plantar pressure reduction at and around the wound site.

A conforming layer may also be provided which is secured to the off-loading layer at the surface opposite from the cushioning layer. The conforming layer serves as a flexible interface between the off-loading layer and the contact surface engaged by the foot and dressing combination, such as the floor, a foot bed, or other foot gear, etc. The additional flexibility resulting from use of the conforming layer provides greater comfort to the foot surface with which the LRD is engaged, as well as additional off-loading of plantar pressure at and around the wound site.

Optionally, an adhesive layer may be provided on the surface of the cushioning layer for securing engagement with the foot. A peel-off double sided tape or other medically appropriate adhesive may be used to enable convenient application by the patient or medical personnel.

As mentioned above, the thickness of each of the cushioning, off-loading and conforming layers may also be varied. Generally, greater pressure reduction at the wound site is obtained by increasing dressing thickness. The LRD should be of a sufficient overall thickness to provide for substantial off-loading of the wound site during walking, without causing a patient to significantly alter their normal gait pattern. Thus, the ideal thickness enables a patient to maintain a steady gait during walking, while allowing maximum off-loading of pressure from the wound site with minimal pressure gain across the surface area of the LRD.

Other features and advantages of the LRD of the present application include the ability of the dressing to accommodate the delivery of adjunct wound therapies to promote healing. For example, an occlusive membrane may be used between the layers of the LRD to create a “well” in connection with the wound opening. Various wound healing therapies, such as antibiotics, antimicrobials, growth factors and cell based therapies, and exudate absorbants, may be delivered to the wound via placement within the well.

Improved Foot Bed

Additionally, an improved foot bed of the present application, or commercially available foot gear having foot beds, may also be used in conjunction with the LRD of the present application. The improved foot bed design includes multiple layers of materials of sufficient thickness to allow the LRD to automatically conform or mold into the foot bed, while also providing firm support for that portion of the foot plantar surface which is not engaged with the LRD. The foot bed further serves to maintain the LRD properly positioned on the foot, and reduces potential shifting of the LRD with respect to the wound site.

The layers of the improved foot bed may include: a top layer for engagement with the LRD; a middle layer having a “landing zone” portion which preferably employs a visco-elastic material to automatically conform or mold around the LRD as it is pressed under load into the landing zone, and has a thickness at least as thick as the LRD, and may also include a support zone portion of a somewhat firmer material; and a bottom layer to provide a rigid surface for supporting the foot bed. In the middle layer, the landing zone portion would be provided substantially in the area of the foot where the wound is present, with the support zone being provided in the other areas of the foot. Alternatively, the improved foot bed may be provided without a top layer. In a preferred embodiment, the visco-elastic material of the landing zone of the middle layer minimizes potential shifting of the LRD by conforming or molding around the LRD geometry.

The improved foot bed geometry or external shape is adapted for engagement with the desired foot gear. The combination of the improved LRD with the improved foot bed, preferably used within appropriate foot gear, provides an improved wound healing system for significant plantar pressure reduction at the wound site.

Other features and advantages will become apparent from the following detailed description and from the accompanying drawings.

### BRIEF DESCRIPTION OF THE DRAWINGS

FIG. 1 illustrates a perspective view of one of the embodiments of the improved load relieving dressing of the present application.

FIG. 2a-2b illustrates a sample of the pressure distributions on the various regions of a foot during load bearing, such as walking.

FIGS. 3a-3c illustrate schematic perspective views of the improved load relieving dressing of the present application for the metatarsal head region, heel region and hallux region, respectively.

FIGS. 4a-4c illustrate side views of the improved load relieving dressing of the present application having various thicknesses in various layers.

FIGS. 5a-5d illustrate the surface of the improved load relieving dressing for engagement with a foot having an adhesive layer; in 5a-5b with a covered wound opening and in 5c-5d with an open wound opening.

FIG. 6 illustrates a partial perspective view of a wound opening having a membrane layer formed between layers of the load relieving dressing of the present application, and forming a well having exudate absorbing material supported therein.

FIGS. 7a-7c illustrate bottom, side and wrapped views of a foot of a patient supporting a load relieving dressing of the present application.

FIGS. 8a-8d illustrate perspective views of various embodiments of the improved foot bed of the present application.

FIGS. 9a-9c illustrate use of the improved foot bed with the improved load relieving dressing, where FIG. 9a shows the approximate position of the improved LRD with respect to the foot bed during use, FIG. 9b shows use of improved foot bed engaged with the improved LRD a wrapped foot of a patient and the landing zone portion conforms to the LRD, and FIG. 9c shows the memory characteristic of the material used in the landing zone portion of the LRD.

FIGS. 10a-10b show the wound healing system of the present application with the improved LRD and improved foot bed on a foot of a patient within conventional foot gear.

FIGS. 11a and 11b illustrate use of the improved LRD of the present application used with one embodiment of a commercially available insole.

FIGS. 12a and 12b illustrate use of the improved LRD of the present application used with another embodiment of a commercially available insole.

### DETAILED DESCRIPTION OF THE INVENTION

The load-relieving dressing and foot bed described here have been shown to provide mechanical off-loading by reducing foot plantar surface pressure at the wound site during load bearing. The dressing and foot bed combination are simple for health care practitioners to apply, easy for patients to use and create a more favorable environment for wound healing.

Load Relieving Dressing

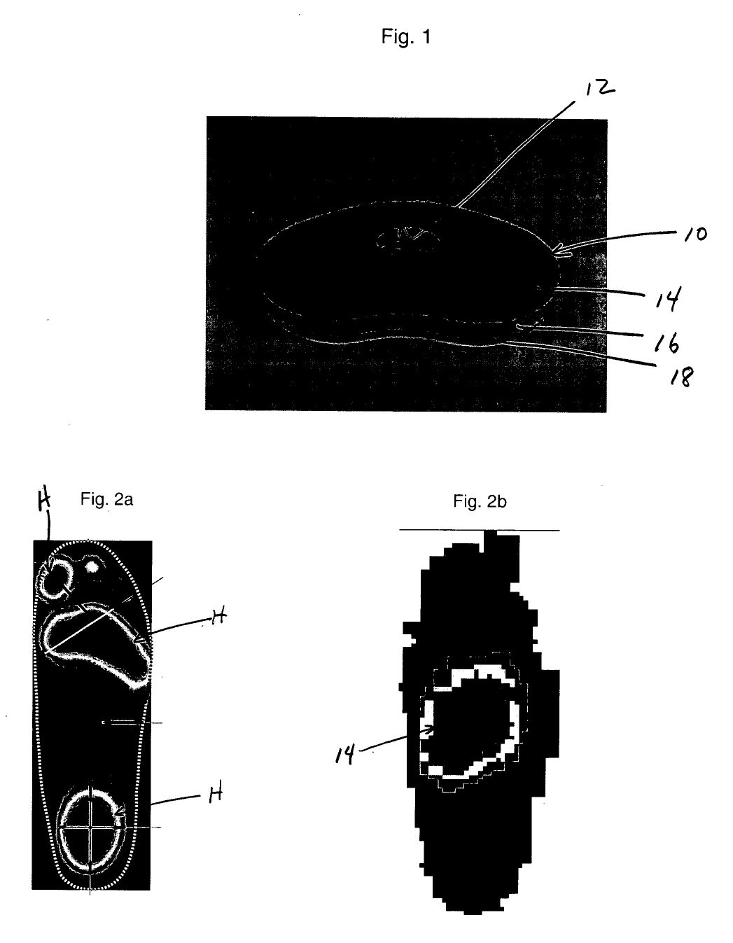

Referring now to the drawings, FIG. 1 shows a load relieving dressing 10 or “LRD” of the present application. The LRD of the present application is provided with an external dressing geometry which is shaped to correspond approximately and substantially to the typical load bearing regions of the foot. FIGS. 2a and 2b demonstrate barefoot pressure distributions, with higher plantar pressures being measured in the highlighted regions indicated at H. Obviously, plantar pressures within the load bearing regions of the foot may vary greatly. In FIG. 2a, the highlighted areas of increased pressure during load bearing include areas of the forefoot, for example, the metatarsal head and hallux regions, as well as the heel area. While in FIG. 2b, measurements of the midfoot area illustrate areas of increased pressure.

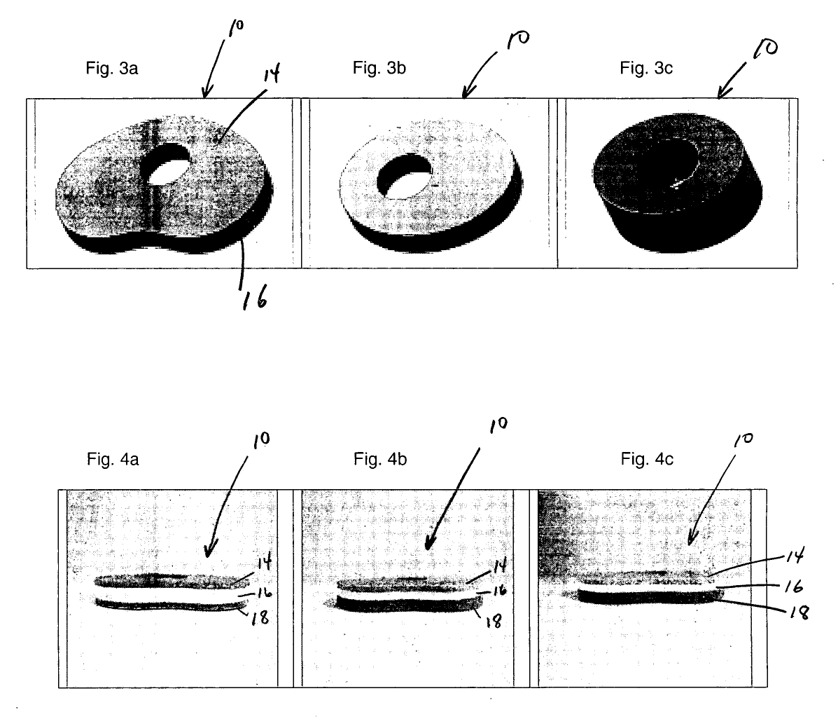

The external geometry of the LRD of the present application preferably covers essentially the increased pressure or weight-bearing anatomy of the foot by region. Thus, in FIG. 3a, the LRD has a substantially lobular configuration for engagement with the pressure distribution in the metatarsal head region of the forefoot. In FIG. 3b, an LRD having an external geometry for use with the pressure distribution in the heel region is illustrated having an ovoid configuration. In FIG. 3c, an LRD having an external geometry for use with pressure distribution in the hallux region is illustrated as ovoid.

The LRD includes a wound opening 12 or off-loading aperture through the LRD for surrounding a wound site, the location and diameter of which aperture is varied based upon the presentation of the wound or wound sites. The wound opening may be formed by conventional stamping methods and may be provided in single or multiple standard locations, such as central, right and/or left locations. When the opening of the LRD is formed or modified by a health care practitioner specifically for a patient, the preferred method of formation or modification is by appropriate cutting instruments such as a scalpel or scissors. The load relieving dressings of the present application are also interchangeable between feet and may be used on either the left foot or the right foot, since rotation of the dressing enables alignment of the dressing with the pressure distribution surface of either foot and/or the wound location.

Additionally, the LRD 10 of the present application is preferably of multiple material layers of foam material, where certain of the layers are of off-loading or load relieving foams. It is preferred that the LRD have an overall thickness in the range of approximately 3 to 25 mm of foam material. The preferred thickness of the LRD enables a patient to maintain a steady gait during walking, while allowing maximum off-loading of pressure from the wound site during load bearing.

A first layer 14 of the LRD is for interfacing with the plantar surface of the foot, and cushioning the foot, particularly at the periphery of the wound opening, during load bearing. The first cushioning layer 14 is preferably of a material sufficient to limit pressure increases at the wound opening and within the dressing at the wound opening, such as a polyurethane foam material. The cushioning layer 14 has a durometer of between 5-30 Shore A hardness. The thickness of the cushioning layer 14 is between 0 to 12 mm.

A second off-loading layer or load relieving layer 16 is engaged with the first cushioning layer 14 of material. The off-loading layer 16 is sufficiently thick and firm to reduce plantar pressure at and around the wound site. The firmer material preferably used for the off-loading material layer is an ethylene vinyl acetate foam material. The off-loading layer 16 has a durometer of between 15-60 Shore A hardness. The thickness of the off-loading layer 16 is between 0 to 12 mm. As illustrated in FIG. 1 and FIG. 4a, the off-loading layer is preferably thicker than the cushioning layer. As illustrated in FIG. 3a, the LRD may be provided with only a cushioning layer 14 and an off-loading layer 16.

An optional third conforming layer 18 is engaged with the off-loading layer 16. The inclusion of a third conforming layer provides several unexpected benefits. A third layer allows reduction in the thickness of the off-loading layer 16, because the conforming layer 18 also provides pressure off-loading at the wound site. As a result, the LRD provides comparable pressure off-loading, but is more flexible, so it better conforms to the plantar aspect of the foot. The conforming layer is of a material sufficient to provide a flexible interface with a contact surface, such as the floor, a foot bed, or other foot gear, etc., being engaged by the foot during load bearing or walking, and thus provides better traction during walking. The material of the conforming layer 18 is sufficient to conform to a foot surface during load bearing conditions for providing additional off-loading of pressure from the wound during load bearing. The conforming layer reduces impact stresses during load bearing, and retains or regains its dimensional properties under non-load bearing conditions, meaning it rebounds to its original shape regardless of the number of weight-bearing cycles that are applied. Materials to be used for the conforming layer include polyurethane foam materials having a durometer of 5-30 Shore A hardness, and a thickness of 0 to 12 mm. In a preferred embodiment, visco-elastic materials are used to improve the overall flexibility of the LRD, and increase traction during walking due to its deformation under load bearing conditions.

As shown in FIGS. 4a, 4b and 4c, the thicknesses of individual layers may be varied to obtain the desired configuration of maximum pressure reduction for a particular patient. The preferred optimal thicknesses illustrated in FIG. 4a are, for example, are: cushioning layer 14: approximately 3 mm, off-loading layer 16: approximately 6 mm and conforming layer 18: approximately 3 mm.

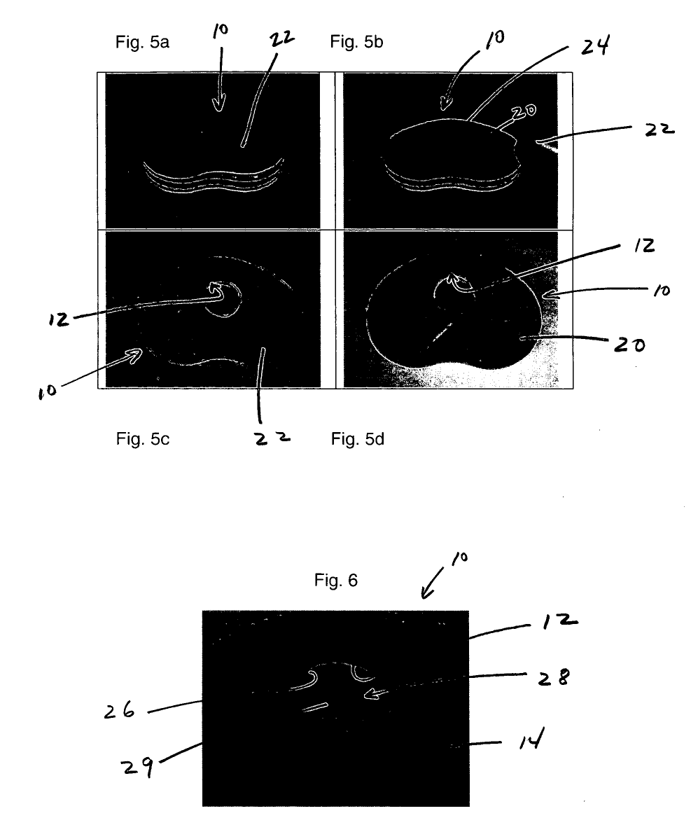

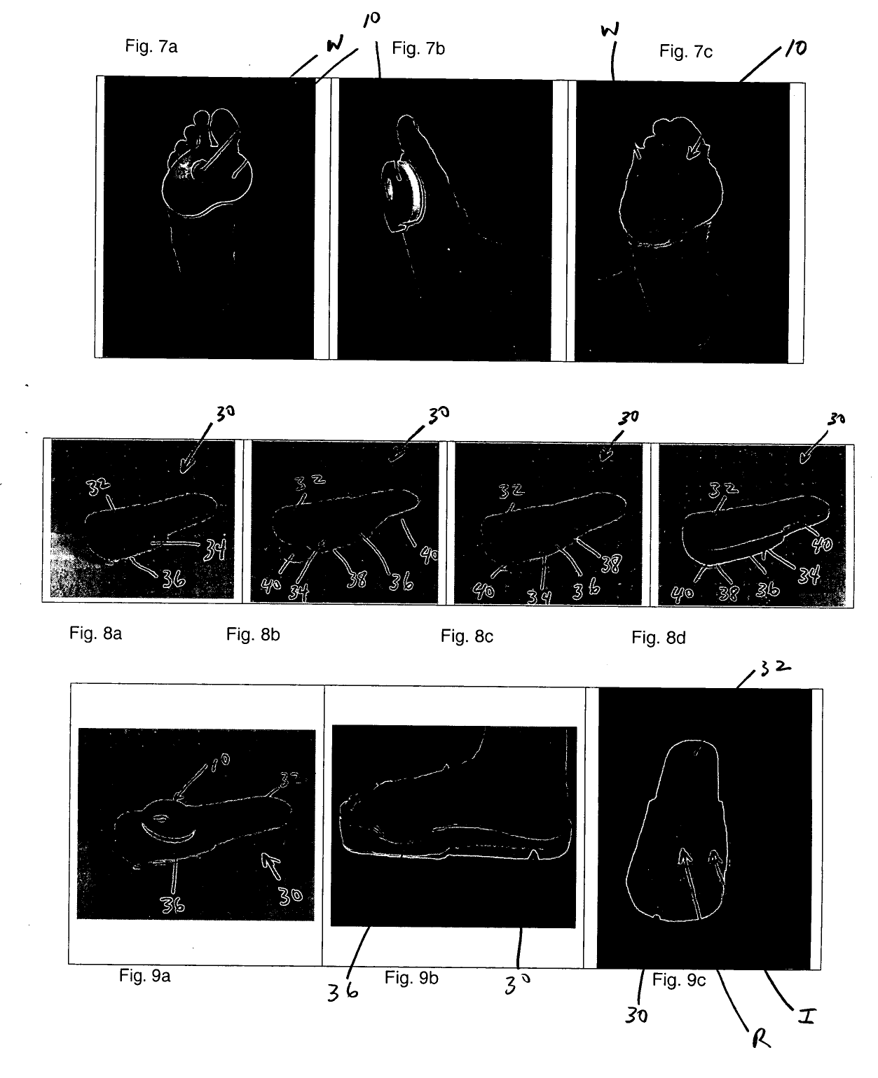

A further optional adhesive layer 20 may also be provided on the surface of the cushioning layer for securing engagement of the LRD with the foot. A peel-off double sided tape or other medically appropriate adhesive, such as Duoderm®, Tegasorb®, Allevyn®, or other adhesives by ConvaTec, for example, may all be used. In FIGS. 5a and 5c, an LRD is illustrated prior to removal of the peel-off adhesive backing 22. FIG. 5b illustrates the LRD with the peel-off backing removed, but with a portion 24 of the adhesive remaining covering the wound opening 24. Such an application may be desired where the adhesive layer applied to the skin over the wound is an exudate absorbent, such as Versiva® by ConvaTec. In FIGS. 5c and 5d, the illustrated LRD has an adhesive layer 20 which also includes a wound opening 12 therethrough. FIGS. 7a and 7b illustrate the LRD applied to a patient's foot using an adhesive layer, and with the wound opening 12 aligned over the wound W. In FIG. 7c, the LRD is further secured to the foot using a self-adhering wrap or other bandage.

In the illustrated LRD of FIG. 6, an occlusive membrane layer 26 is provided between the cushioning layer 14 and off-loading layer 16. As shown in FIG. 6, a “well” or cavity 28 is formed within the wound opening and membrane layer 26 to support a desired wound healing therapy. In the illustrated embodiment of FIG. 6, Aquacel® wound dressing by ConvaTec is provided within the well 28. A variety of therapies may be placed within the well for delivery to the wound, such as antibiotics, antimicrobials, growth factors and cell based therapies, and other exudate absorbants. This may be accomplished either by incorporation of a membrane layer in the dressing 10 or by custom packing of the LRD wound aperture 12 by the healthcare practitioner.

Manufacture of the LRD of the present application includes the steps of laminating the desired foam material layers in the desired order to achieve the preferred thickness. Next the foam layers may be machined to the desired external geometry and to form a wound opening. A layer of adhesive material may next be applied. Finally, any desired wound healing therapies may be incorporated into the dressing.

Foot Bed and Wound Healing System

FIGS. 8a to 8d illustrate an improved foot bed of the present application. While the use of the LRD alone is intended and provides increased wound healing, typical wound healing practices generally include a protective insole and related foot gear. However, the effectiveness of such foot gear is dependent upon patient compliance during heightened risk for injury to the wound site. In the present application, the preferred embodiment of the improved LRD is provided for use in an improved foot bed.

In the improved foot bed 30 of the present application, a material layer is provided which has sufficient thickness to allow the LRD to automatically conform or mold into the foot bed. A material layer providing firm support for the foot plantar surface not engaged with the LRD may also be provided. The foot bed of the present application maintains the LRD properly positioned on the foot, and reduces shifting of the LRD with respect to the wound site as shown in FIG. 9b. The external geometry of the foot bed is configured for engagement within any desired outer foot gear.

In the preferred embodiments of the present application, three or four material layers are provided. In the three layer embodiments of FIG. 8a:

a top layer 32 is provided for engagement with the LRD 10 (as shown in FIG. 9a) which is manufactured of polyurethane material and of a thickness of approximately 1 mm to 6 mm;

a middle layer 34 having a “landing zone” portion 36 which preferably employs a visco-elastic material of the type available from suppliers of Tempur-pedic® or Tempur-Med® products, to automatically conform or mold around the LRD 10 as the LRD is pressed under load into the landing zone. It is desired that the landing zone of the middle layer be of a material which may be compressed to a thickness enabling the surface of the foot on which the LRD is secured to be approximately level with the surface of the foot bed. Thus, the LRD and foot bed provide a surface which enables a normal walking gait, and which supports the foot equally in load bearing and non-loading sections. The middle layer has a thickness approximately at least as thick as the LRD, or between 10 mm and 30 mm, and more preferably approximately 20 mm; and

a bottom layer 38 of a more rigid material, for example, ethylene vinyl acetate (“EVA”) material, to provide a rigid surface for supporting the foot bed 30, and of a thickness of approximately 1 mm to 6 mm. Thus, the overall thickness of the foot bed may range from 12 mm to 42 mm, and preferably approximately 30 mm.

In the four material embodiment of FIGS. 8b, 8c and 8d, an additional support zone portion 40 of a somewhat firmer material such as EVA, or combinations of EVA and visco-elastic material, may also be provided. The landing zone portion would be provided in the area of the foot where the wound is present, with the support zone supporting other areas of the foot not engaged with the LRD 10, as shown in FIGS. 8b and 8d.

As shown in FIG. 9c, the top layer 32 and landing zone 36 f the middle layer 34, conform to or mold around the LRD 10. As shown, the combination of the LRD 10 within the foot bed 30 serves to relieve pressure or off-load the wound W during load bearing, which can be seen by the indentation I formed in the foot bed 30 by the LRD and the relief section R where the wound opening 12 is located within the LRD adjacent the wound.

Manufacture of the improved foot bed of the present application includes the steps of laminating the desired material layers in the desired order to achieve the preferred thickness and characteristics. Alternatively, layers of materials may be co-extruded or cut and glued together to form the desired patterns within the middle layer 34.

The improved LRD 10 of the present application may also be used with numerous conventional foot beds. As shown in FIGS. 11, the LRD 10 is used with a Don Joy foot bed 30′ having two layers of material. In FIGS. 12 the LRD 10 is shown engaged within a Royce Medical insole 30″, with the hexagonal plugs 31 of the insole removed to accommodate the external geometry of the LRD 10. Additionally, where commercially available one layer foot beds are used, thermoformed materials such as Plastizote (not illustrated) may be used with the present LRD 10.

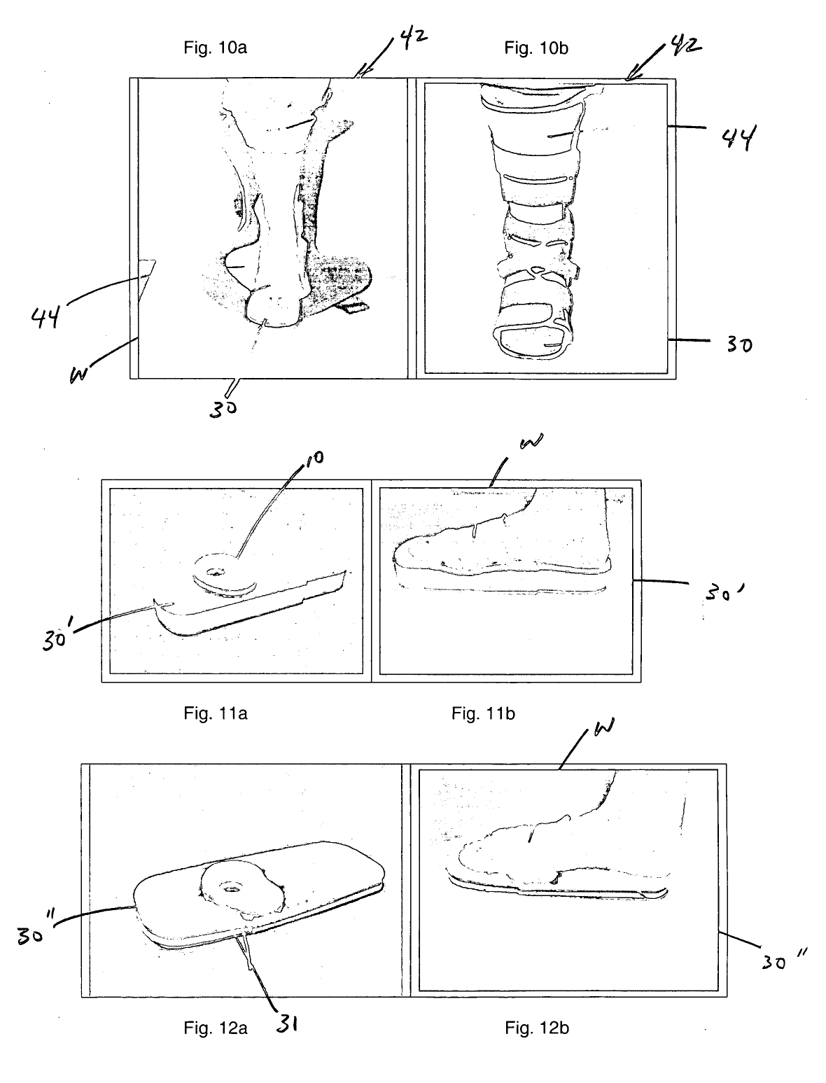

The improved foot bed 30, LRD 10 and foot gear 44 combination, or the wound healing system 42 of the present application, are shown in FIGS. 10a and 10b. The combination of the improved load relieving dressing 10 with the improved foot bed 30, preferably used within appropriate foot gear 44, provides an improved wound healing system 42 for significant plantar pressure reduction at the wound site.

While different embodiments of the invention have been described in detail herein, it will be appreciated by those skilled in the art that various modifications and alternatives to the embodiments could be developed in light of the overall teachings of the disclosure. Accordingly, the particular arrangements are illustrative only and are not limited to the scope of the invention which is to be given the full breadth of any and all equivalents.

We claim:

1. A load relieving dressing for transferring pressure away from a wound on a foot of a patient comprising:
a wound opening formed through said dressing for surrounding a wound site;
a first layer of said dressing for interfacing with and cushioning a foot during load bearing, said first layer of a material sufficient to limit pressure increases at said wound opening and within said dressing adjacent said wound opening;
an off-loading layer engaged with said first layer of a material sufficiently thick and firm to reduce pressure at a wound site;
a conforming layer engaged with said off-loading layer and of a material sufficient to provide a flexible interface with a contact surface being engaged by a foot during load bearing, said material sufficient to conform to a foot surface during load bearing conditions, provide additional off-loading of pressure from a wound during load bearing, reduce impact stresses during load bearing, and to retain dimensional properties under non-load bearing conditions.

2. A load relieving dressing for transferring pressure away from a wound on a foot of a patient comprising:
a wound opening formed through said dressing for surrounding a wound site;
a first layer of said dressing for interfacing with and cushioning a foot during load bearing, said first layer of a material sufficient to limit pressure increases at said wound opening and within said dressing adjacent said wound opening; and
an off-loading layer engaged with said first layer of a material sufficiently thick and firm to reduce pressure at a wound site.

3. The load relieving dressing of claims 1 or 2, wherein said first layer further supports an adhesive layer positioned intermediate said first layer and a foot of a patient for securing said load relieving dressing to a foot.

4. The load relieving dressing of claims 1 or 2, wherein said dressing has a geometry shaped substantially to the pressure distribution on a foot of a patient during load bearing.

5. The load relieving dressing of claim 4, wherein said wound opening may be positioned through any portion of said dressing for appropriately surrounding a wound site.

6. The load relieving dressing of claims 1 or 2, wherein said dressing has a lobular geometry.

7. The load relieving dressing of claims 1 or 2, wherein said dressing has a geometry which is substantially ovoid in configuration.

8. The load relieving dressing of claim 5, further comprising a membrane layer positioned intermediate two adjacent layers of said dressing, said membrane layer supporting wound healing therapy materials at a location adjacent a wound for promoting wound healing.

9. A load relieving dressing for transferring pressure away from a wound on a foot of a patient comprising:
a wound opening formed through said dressing for surrounding a wound site;
said dressing substantially engaging a surface of a foot of a patient which supports a pressure distribution of a foot of a patient during load bearing;
said dressing having a shape which substantially conforms to the shape of the pressure distribution on the foot during such load bearing; and
said wound opening may be positioned anywhere within said dressing for appropriately surrounding a wound site.

10. A wound healing system for healing a wound on a foot of a patient comprising:
a load relieving dressing to be attached to an area about the wound of the foot, whereby said load relieving dressing provides support to the foot in said area and relieves load on the wound and includes a wound opening surrounding the wound; and
a foot bed for engagement with a foot of a patient having said load relieving dressing, said foot bed having a base portion and a landing zone portion, said landing zone portion for engagement with said load relieving dressing, and having a thickness which is approximately equal to or greater than the thickness of the load relieving dressing, and of a material sufficiently compressible to fully conform to said load relieving dressing during load bearing, said base portion supporting said landing zone portion and providing a rigid base for supporting a patient during load bearing.

11. The wound healing system of claim 10, wherein said foot bed is engaged within foot gear worn by a patient.

12. The wound healing system of claim 10, wherein said landing zone portion of said foot bed is positioned substantially within a metatarsal head region of a foot of a patient, said landing zone portion positioned adjacent a support zone portion secured to said landing zone portion and located on proximal and/or distal ends of said landing zone portion, said support zone portion of a firm material for supporting portions of the foot not engaged with said load relieving dressing, and said base portion supporting said landing zone portion and support zone portion.

13. An improved foot bed for transferring pressure away from a wound on a foot of a patient comprising:
a base portion having a planer surface adapted to be under a foot and of a sufficiently rigid material for supporting said foot bed within appropriate foot gear or for engagement with the floor during load bearing by a patient;
said base portion supporting a landing zone portion thereon, said landing zone portion for engagement with a load relieving dressing engaged on a foot of a patient, and having a thickness which is approximately equal to or greater than the thickness of the load relieving dressing, and of a material sufficiently compressible to fully conform to said load relieving dressing during load bearing; and
said base portion supporting a support zone portion positioned to surround said landing zone portion for supporting portions of a foot of a patient which are not engaged with a load relieving dressing.

14. The improved foot bed of claim 13, wherein the landing zone portion is of a visco-elastic foam material.

15. A load relieving dressing for transferring pressure away from a wound on a foot of a patient comprising:
a wound opening formed through said dressing for surrounding a wound site;
said dressing substantially engaging a surface of a foot of a patient which supports a pressure distribution of a foot of a patient during load bearing;
said dressing having an external shape which substantially conforms to the shape of the pressure distribution on the foot during such load bearing;
said dressing having a first layer for interfacing with and cushioning a foot during load bearing, said first layer of a material sufficient to limit pressure increases at said wound opening and within said dressing adjacent said wound opening; and
said dressing having a base layer of material sufficiently thick and firm to reduce pressure at a wound site and to provide a flexible interface with a contact surface being engaged by a foot during load bearing, said base layer conforming to a foot surface during load bearing conditions, providing additional off-loading of pressure from a wound during load bearing, reducing impact stresses during load bearing, and retaining dimensional properties under non-load bearing conditions.

16. The load relieving dressing of claim 15, wherein said first layer further supports an adhesive layer positioned intermediate said first layer and a foot of a patient for securing said load relieving dressing to a foot.

17. The load relieving dressing of claim 15, wherein said dressing is interchangeable and may be used on either a right foot of a patient or a left foot of a patient.

18. The load relieving dressing of claim 17, wherein said dressing has a lobular geometry.

19. The load relieving dressing of claim 17, wherein said dressing has a geometry which is substantially ovoid in configuration.

20. The load relieving dressing of claim 15, further comprising a membrane layer positioned intermediate two adjacent layers of said dressing, said membrane layer supporting wound healing therapy materials at a location adjacent a wound for promoting wound healing.
